(19) United States
(12) Patent Application Publication
(10) Pub. No.: US 2009/0177102 A1
(43) Pub. Date: Jul. 9, 2009

(54) SYSTEM, METHOD AND DEVICE FOR PREDICTING SUDDEN CARDIAC DEATH RISK

(75) Inventors: Mary Schneider, Brookfield, WI (US); Patrick Dorsey, Menomonee Falls, WI (US); Joel Schoenbeck, West Bend, WI (US)

Correspondence Address: Andrus, Sceales, Starke & Sawall, LLP, 100 East Wisconsin Avenue, Suite 1100, Milwaukee, WI, 53202-4178, US

(73) Assignee: The General Electric Company, Schenectady, NY (US)

(21) Appl. No.: 11/970,314

(22) Filed: Jan. 7, 2008

(51) Int. Cl. A61B 5/0452

(52) U.S. Cl. 600/516

(57) ABSTRACT

A system and method for predicting sudden cardiac death. The system includes a patient monitoring station, a Holter analysis workstation, and a hospital information network. The Holter analysis workstation being operative to apply a plurality of data analysis algorithms to create a sudden cardiac death report. The method applies a first data analysis technique and a second data analysis technique to electrocardiographic data to produce an indication of sudden cardiac death risk.

### FIELD OF THE DISCLOSURE

The present disclosure relates to the field of monitoring the physiological condition of a patient. More specifically, the present disclosure relates to analyzing the risk of a patient suffering from sudden cardiac death.

### BACKGROUND

Sudden cardiac death (SCD) is a leading cause of death in adults. One of the greatest threats of sudden cardiac death is that the effects and symptoms are sudden and unexpected. SCD may often occur within minutes after the symptoms first appear. While an underlying heart condition, such as atherosclerosis or a previous heart attack, may increase a patient's risk of SCD, some victims may be children or have no prior history of heart disease.

SCD occurs when the electrical impulses generated by the heart and propagated through the heart muscle tissue become rapid (tachycardia) or chaotic (fibrillation) or both. The physiological events leading up to sudden cardiac death may be triggered by an irregular heart rhythm (arrhythmia), the body's inability to control tachycardia, or the extreme slowing of the heart (bradycardia).

Current monitoring for SCD is performed by a retroactive review of previously recorded patient electrocardiographic (ECG) data. Many SCD monitoring algorithms require ECG data acquired over a period of time to perform an accurate analysis. Therefore, sudden cardiac death monitoring systems and methods often use a portable ECG recording device that is worn by the patient for a duration of time, usually spanning between 12 and 72 hours. During this period of time the monitoring device records the patient's ECG data and at the end of the test, the ECG data is downloaded from the device to a computer such that the patient's risk of sudden cardiac death may be determined by analyzing the ECG data.

The resulting sudden cardiac death risk analysis is a retrospective report of the patient's condition over the past 12-72 hours. This leads to a reactionary response by the clinician to the previously collected data. Such a system where the responses are reactionary can be detrimental to patient care, since the patient may have already been discharged from the hospital or begun treatment and/or procedures that are adverse to a condition of elevated sudden cardiac death risk.

### BRIEF DISCLOSURE

In the field of patient monitoring it is desirable to have a system, method, and device that monitors physiological data collected from a patient and produces a prediction of a patient's risk of sudden cardiac death. Embodiments of the system disclosed herein may include a patient monitoring station that acquires at least electrocardiographic data from a patient. A Holter analysis workstation may be communicatively connected to the patient monitoring station such that the Holter analysis workstation acquires at least electrocardiographic data from the patient at predetermined time intervals. The Holter analysis workstation may then apply data analysis algorithms to the electrocardiographic data to create a sudden cardiac death report. A hospital information network communicatively connects clinicians with the Holter analysis workstation such that at least one clinician is notified of the sudden cardiac death report.

Embodiments of a Holter analysis device with sudden cardiac death risk analysis capability are also disclosed herein. These embodiments may include an electrocardiographic data retrieval module. The data retrieval module retrieves electrocardiographic data that has been acquired over a predetermined time period. The Holter analysis device may further include a first sudden cardiac death analysis technique module. The first technique module produces a first indication of sudden cardiac death risk. The Holter analysis device further includes a second cardiac death analysis technique module. The second technique module produces a second indication of the sudden cardiac death risk. Finally, the Holter analysis device may include a sudden cardiac death report generation module that receives the first and second indications of sudden cardiac death risk and produces a sudden cardiac death report based upon the first and second indications.

Embodiments of a method of predicting a patient's risk of sudden cardiac death are also disclosed herein. Embodiments of this method include receiving electrocardiographic data from a patient and applying a first electrocardiographic data analysis technique to the electrocardiographic data. The method further includes applying a second electrocardiographic data analysis technique to the electrocardiographic data to produce a second indication of sudden cardiac death risk. Further embodiments of the method may include analyzing the first indication of sudden cardiac death risk and the second indication of sudden cardiac death risk to produce a composite indication of the patient's risk of sudden cardiac death.

### BRIEF DESCRIPTION OF THE DRAWINGS

FIG. 1 is a schematic diagram of an embodiment of a system for predicting sudden cardiac death;

FIG. 2 is a flow chart depicting the steps of an embodiment of a method for predicting sudden cardiac death risk;

FIG. 3 is a flow chart depicting a more detailed embodiment of the application of sudden cardiac death risk algorithms; and

FIG. 4 is a flow chart depicting an embodiment of a method of an ECG management system workflow.

### DETAILED DISCLOSURE

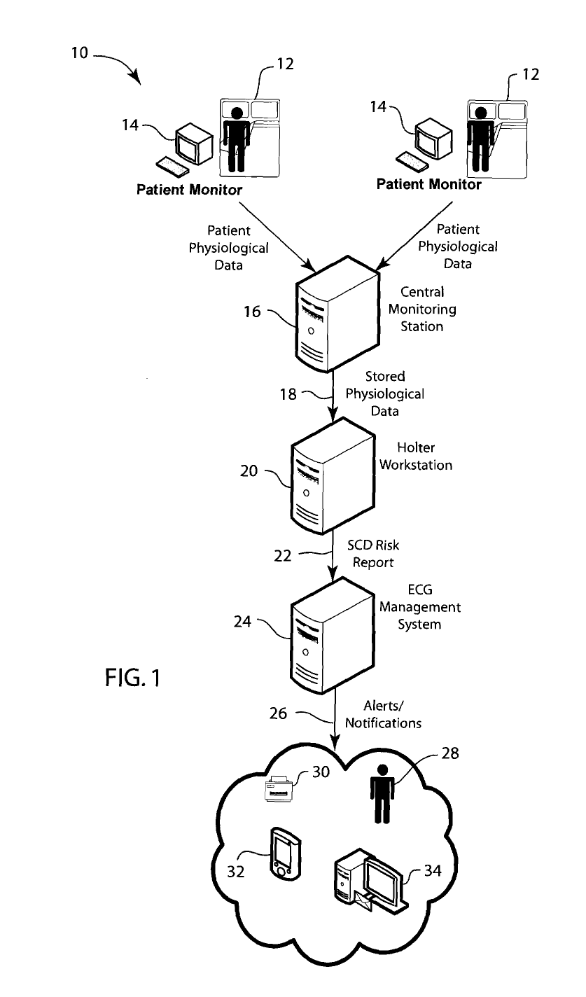

FIG. 1 depicts an embodiment of a patient monitoring system 10. The patient monitoring system 10 includes one or more patients 12 connected to a patient monitor 14. The patient monitor 14 may be attached to the patient via a plurality of electrodes (not depicted) or other transducers (not depicted) that collect a variety of physiological data from the patient. The physiological data may be collected by wired or wireless transmission from the transducers to the patient monitor 14.

The collected physiological signals may include electrocardiographic (ECG) data, respiration rate, blood pressure, and SpO₂. Additional physiological data collected by the patient monitor 14 may include arterial pressure (ART), central venus pressure (CVP), intracranial pressure (ICP), pulmonary artery pressure (PA), left arterial pressure (LA), special pressure (SP), femoral arterial pressure (FEM), right arterial pressure (RA), umbilical arterial pressure (UAC), umbilical venus pressure (UVC), cardiac output (CO), carbon dioxide (CO2) and end tidal carbon dioxide (ETCo2), and electroencephalograph (EEG). It is understood that other physiological data known to one skilled in the art may also be collected by the patient monitor 14. At a minimum, the patient monitor 14 collects ECG data from the patient 12. The ECG data may include the standard twelve lead ECG waveform data and may be sampled at a rate between 120 Hz and 240 Hz; however these specifications are merely exemplary as to the ECG monitoring performed by the patient monitor 14.

The patient monitor 14 collects the physiological data from the patient 12 in real time and transmits the collected physiological data to a central monitoring station 16 in real time. The central monitoring station 16 receives the physiological data from a plurality of patient monitors 14, which may include all of the patient monitors 14 in a particular region of a hospital or other medical facility such as a floor or wing of the medical facility. The transmission of the physiological data from the patient monitors 14 to the central monitoring station 16 may be performed via a wired connection or a wireless connection. Preferably, the physiological data transmission will be in real time as it is collected by the patient monitor; however, the data transmission may alternatively be periodic or multiplexed between the various patient monitors 14.

The central monitoring station 16 receives the collected patient physiological data and stores the data for later retrieval and/or processing. Additionally, the central monitoring station 16 may perform some signal processing and/or administrative function with the patient physiological data. These functions may include correlating the physiological data with a patient's electronic medical record (EMR) and/or storing the collected physiological data in the proper locations within the healthcare provider's IT network.

Next, the stored physiological data 18 is transmitted to a Holter workstation 20. The Holter workstation 20 receives the physiological data 18 and applies a variety of signal processing techniques to the physiological data 18. In one embodiment, the data processing techniques include one or more sudden cardiac death prediction algorithms, as will be described in further detail herein. As a result of the application of the one or more sudden cardiac death algorithms, the Holter workstation 20 produces an SCD risk report 22. The SCD risk report 22 includes the results or outputs of the application of one or more SCD algorithms to the physiological data. The SCD report generally provides an indication of a patient's risk of sudden cardiac death. The indication of risk may be a percentage or other indication of the likelihood of occurrence of sudden cardiac death or a more generalized characterization of risk such as a gradation comprising “low,” “medium,” and “high” designations.

The SCD risk report 22 is sent from the Holter workstation 20 to an ECG management system 24. The ECG management system 24 provides additional processing of the SCD risk report and coordinates the alert and/or notification of one or more clinicians of the results of the SCD risk report. The ECG management system 24 preferably provides an alert or notification 26 to a variety of communication devices associated with a clinician 28. The alerts and/or notifications 26 may be sent to a printer and/or fax machine 30, a personal digital assistant (PDA) 32 that is carried by the clinician and/or in close proximity to the clinician 28, and/or a computer workstation 34 at which the clinician 28 receives notifications such as through emails and/or through other instant messaging communication techniques.

Alternatively, the ECG management system 24 may not be necessary in embodiments of the patient monitoring system 10 as disclosed herein. In those embodiments, the Holter workstation 20 may be connected to a hospital information network. The hospital information network includes but is not limited to one or more information servers (not depicted) connected via wired and wireless connections to a variety of computer workstations, clinician PDA's, mobile computer devices, and/or other communication devices associated with one or more clinicians such that digital information stored in the one or more servers is accessible to the one or more clinicians. The SCD risk report 22 may be transmitted via the hospital information network to one or more of the communication devices in association with the clinician 28. In these such embodiments, the Holter workstation 20 may include additional processing such that the SCD risk report 22 is in a format suitable for delivery to the communication devices and/or to include an identification of the particular clinicians to which the SCD risk report 22 is to be sent.

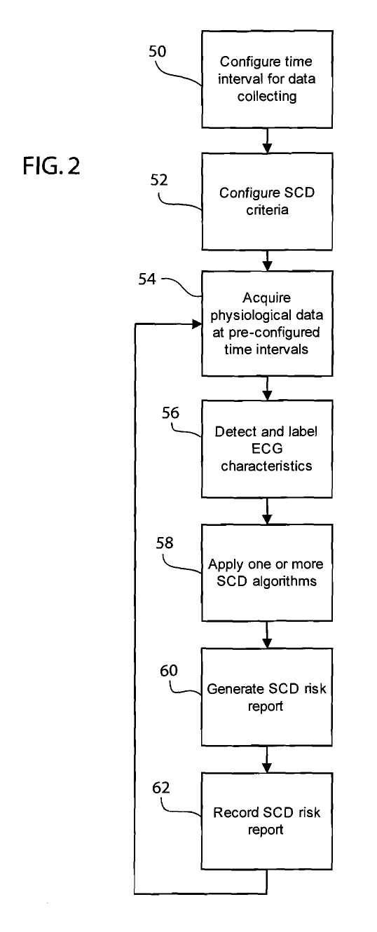

FIG. 2 depicts an embodiment of a method carried out by the embodiments of the Holter workstation 20. First, at step 50 the time interval for data collection is configured. In this step, the period of time between the acquisitions of stored physiological data is set by a clinician or a program or module interval to the Hotter workstation. While physiological data may be collected from the patient in real time, the Holter workstation may only acquire the collected physiological data at set time intervals. These time intervals may range from a minute or less of physiological data to one or more hours of physiological data. In an alternative embodiment, the Holter workstation receives patient physiological data in real time; however, at step 50 the Holter workstation segments the physiological data into groups based on a set time interval. Next, at step 52, the SCD criteria are configured. The configuration of the SCD criteria may be performed manually by a clinician, but also may be performed by stored computer code as according to a clinician, hospital, or healthcare provider defined set of SCD criteria. The configuration of the SCD criteria may include the selection of one or more SCD risk analysis algorithms to be applied to the acquired physiological data. The SCD risk analysis algorithms are used to calculate a patient's risk of SCD based upon the physiological data.

At step 54, physiological data is acquired at the pre-configured time intervals. The physiological data may be acquired from the patient monitor 14, the central monitoring station 16, or directly from the patients 12 themselves. The physiological data that is acquired typically includes at least electrocardiographic (ECG) data.

Next, characteristics of the ECG data are detected and labeled at step 56. The ECG characteristics include identifying heart beats and labeling the morphological features of the ECG data which may include labeling the QRS complex, the T-wave, or many other ECG morphological features. The detection and labeling of ECG characteristics in step 56 includes the classification of each beat as being normal or abnormal such as being arrhythmic, tachycardic, or bradycardic.

Next, at step 58, one or more SCD algorithms are applied to the physiological data. As will be detailed further herein, there may exist a plurality of SCD algorithms from which the applied algorithms are selected. This selection may be performed by a clinician, or may be part of a predefined procedure as defined by a particular clinician, group of clinicians, hospital, or healthcare provider. Each of the plurality of SCD algorithms analyze different physiological data, or combinations of physiological data or analyze physiological data in specific ways such as to produce different indications of SCD risk.

Then, at step 60, the results from the SCD algorithms applied in step 58 are used to generate an SCD report. The generated SCD report includes a composite risk analysis of the patient risk of SCD based upon the individual results of SCI) risk as computed by the SCD algorithms applied in step 58. Next, at step 62, the SCD report is recorded. The SCD report may be recorded on the ECG management system 24; however, the SCD report may be alternatively transmitted to a communication device that is associated with or in close proximity to an identified clinician such that the SCD report is received and recorded using the communication device. In these embodiments the recorded SCD report may be a print out from a printer or fax or electronically stored on the memory of a PDA or other clinician computer workstation.

After the SCD report has been recorded in step 62, the steps may be repeated, especially the steps starting from step 54 wherein physiological data is acquired at the preconfigured time interval. The physiological data may be acquired at the preconfigured time intervals for the duration of a patient's stay at a hospital or medical care facility, or the physiological data may be acquired from an ambulatory patient for designated time period. In still further embodiments, the physiological data may be acquired at preconfigured time intervals for a long or ongoing time period such as in a situation where a patient is in a remote location, such as his or her home, and being remotely monitored by a clinician at a centralized location.

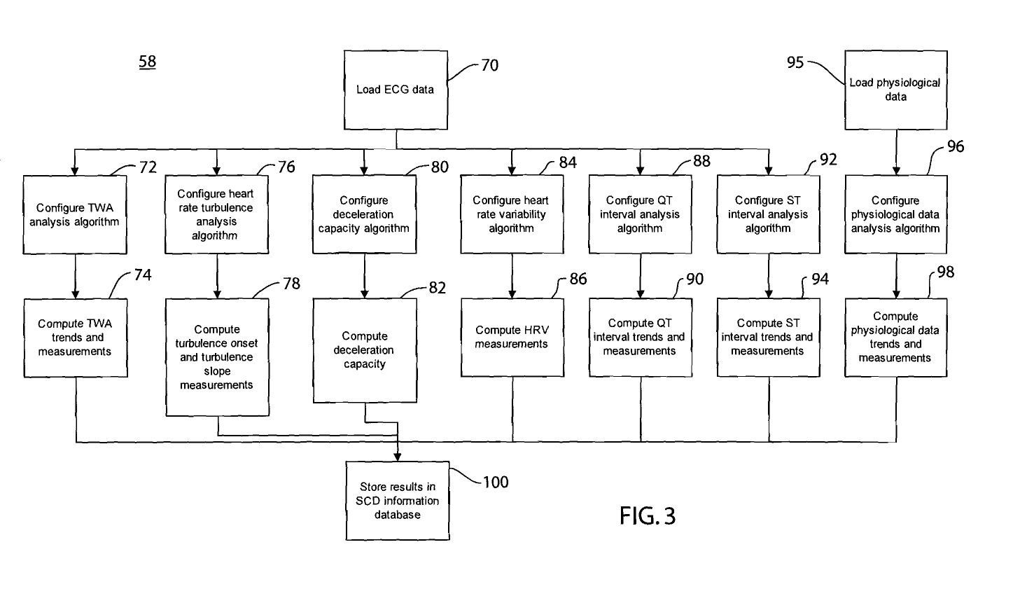

FIG. 3 is a more detailed flow chart of steps followed in an embodiment of step 58 of applying one or more SCD algorithms. In the embodiment depicted in FIG. 3, the physiological data that is analyzed by the SCD algorithms is ECG physiological data that has been processed to detect and label the ECG characteristics as in step 56 depicted in FIG. 2.

First, the ECG data is loaded in step 70 into the computer or system that will apply the SCD algorithms to the ECG data. The loaded ECG data may include the labeled ECG characteristics or other beat annotations or classifications. These labels, annotations, or classifications assist some or all of the SCD algorithms that are applied to the ECG data.

Next, the selected SCD algorithms are applied to the ECG data. The SCD algorithms that are applied include at least one of the algorithms selected from the list of T-wave alternans (TWA) 74, heart rate turbulence 78, and/or heart deceleration capacity 82. While the applied SCD algorithms include at least one of the aforementioned SCD algorithms, this listing is merely exemplary of the types of SCD algorithms that may be applied in step 58. Other alternative SCD algorithms that may be applied in conjunction with one or more of the already identified algorithms include computing heart rate variability, QT interval analysis, ST interval analysis and/or analysis of other physiological data correlated to SCD risk.

Specifically, a T-wave alternans detection algorithm applied by first configuring the TWA analysis algorithm at step 72 and computing the TWA trend and measurements in step 74. An example of TWA alternans detection algorithms that may be used in conjunction with embodiments disclosed herein is disclosed in U.S. Pat. No. 5,148,812 to Verier et al.; however, the algorithms as disclosed therein are merely exemplary of the types of TWA detection algorithms that may be utilized with embodiments as disclosed herein.

Cardiac vulnerability to ventricular fibrillation is dynamically tracked by analysis of alternans in the T-wave and ST segment of an ECG. In TWA detection algorithms, the term “T-wave” may be defined to mean the portion of an ECG which includes both the T-wave and the ST segment. Alternans in the T-wave result from different rates of re-polarization of the muscle cells of the ventricles. The extent to which the cells recover (or re-polarize) non-uniformly is the basis for electrical instability of the heart. TWA detection algorithms provide a method for quantifying cycle-to-cycle variation within the ECG, and particularly the T-wave. Techniques such as Fourier power spectrum analysis, non-linear transformation, spectral analysis, complex demodulation, or dynamic alternation amplitude estimation techniques may be used to quantify the beat-to-beat variance experienced in the patient ECG.

Next, the heart rate turbulence is analyzed through the steps of configuring the heart rate turbulence analysis algorithm 76 and computing the turbulence onset and turbulence slope measurements 78. The step of computing the turbulence onset and turbulence slope measurements includes the construction of the tachogram waveform as these results may help to provide an improved indication of SCD risk depending upon the heart rate turbulence algorithms that are applied to the ECG data. An example of the heart rate turbulence algorithms that may be configured in step 76 and applied in step 78 may include those algorithms disclosed in U.S. Pat. No. 6,496,722 to Schmidt; however, this is not intended to be limiting on the scope of heart rate turbulence algorithms that may be used in conjunction with embodiments as disclosed herein.

Heart rate turbulence is characterized by the existence of extrasystoles which are heartbeats that occur prematurely outside the regular base rhythm. It has been found that extrasystoles leave characteristic signatures in the base rhythm that can be used for risk stratification. For persons with a normal or slightly increased risk, as a rule, the heartbeat sequence following an extrasystole usually accelerates, but only for a few heartbeats, which is then followed by a phase of frequency decrease of the heartbeat sequence. For persons with an increased risk this characteristic reaction is significantly weaker or missing altogether. In these cases, often a more or less erratic heartbeat sequence, that is, one without order or turbulent, can be found. As mentioned above, analyzing the heart rate turbulence requires computing the turbulence onset, the difference of the mean values of the last normal RR intervals preceding the extrasystole and the first normal RR intervals following the extrasystole, and the slope at the greatest frequency decrease within a sequence of several heartbeat intervals. Additionally, the correlation co-efficient of the slope which is a measure for the regularity of the slope may be another relevant value to compute. Each of these quantities has proved suitable for use in determining the patient's sudden cardiac death risk. A small onset, a flat slope, or a low correlation co-efficient of the slope indicates a significantly increased risk of dying in the near term. Alternatively, signal processing in the frequency domain may be used to identify low and high frequency portions of the ECG signal. An increase in the high frequency portions is indicative of an increased risk of dying in the near term.

The deceleration capacity may be determined through the steps of configuring a deceleration capacity algorithm 80 and computing the deceleration capacity 82. The step of computing the deceleration capacity further includes constructing an average waveform that may aid a clinician or analysis program in interpreting the results yielded from the application of the deceleration capacity algorithm to the ECG data. A non-limiting example of an algorithm that may be used to compute the deceleration capacity is disclosed in U.S. Pat. No. 7,200,528 to Schmidt et al.

The deceleration capacity maybe used to evaluate the sudden cardiac death risk of a patient by sequencing the beat-to-beat intervals of the ECG measurement. Next, an attribute may be assigned to each measured value that is equal to the measured value itself divided by the previous measured value. Thus the attribute is representative of each measured interval with respect to the previously measured interval as a percentage of the previously measured interval. The estimation of sudden cardiac death risk in patients may be made by subtracting the sum of the two previously calculated attributes from the sum of a target attribute and the subsequent attribute. This evaluation defines a relationship between the target measured value and the immediately proceeding measured values. The greater the result of this evaluation, the greater the patient's chance of survival as the heart is able to produce and control a greater range of heart rate fluctuations.

In some embodiments, a TWA algorithm, a heart rate turbulence algorithm, and a deceleration capacity algorithm are applied to the ECG data. In other embodiments, two of the aforementioned TWA, heart rate turbulence, and deceleration capacity algorithms are applied to the ECG data. In still further embodiments, only one of these three algorithms are applied to the ECG data and at least one other algorithm is applied to physiological data of the patient. The other algorithms may include heart rate variability, QT interval analysis, ST interval analysis, or any other physiological data analysis that is found to be correlated to SCD risk.

The application of a heart rate variability algorithm to the ECG data includes the steps of configuring a heart rate variability algorithm 84 and computing heart rate variability measurements 88. The application of a QT interval analysis algorithm to the ECG data includes the steps of configuring a QT interval analysis algorithm 88 and computing QT interval trends and measurements 90. Similarly, the application of an ST interval analysis may includes the steps of configuring an ST analysis algorithm 92 and computing ST interval trends and measurements 94.

Additionally, other physiological data collected from the patient 12 by the patient monitor 14 can be incorporated into the analysis and application of the SCD algorithms. This additional physiological data is loaded in step 95 into the computer, system, or software module that will apply any physiological data analysis SCD algorithm. Then, at step 96, at least one physiological data analysis algorithm is configured and then applied in step 98 to compute physiological data trends and measurements.

The results of the application of the selected SCD algorithms to the ECG or other physiological data, these results are stored in step 100 to an SCD information database. These results are then used in step 60 of FIG. 2 to generate the SCD risk report.

The configuring steps as described above include standard data processing functions as would be required to prepare for the application of an algorithm to a set of data. Such configuration includes the selection of one or more algorithms to be applied to the data. The step of configuring includes data processing steps such as the selection of the data to which the algorithms will be applied, the source and/or electronic storage location of the selected data and the initialization of variables within the selected algorithms.

In some embodiments, such as that depicted in FIG. 1, the Holter workstation 20 produces the SCD risk report 22 which is sent to an ECG management system 24. The ECG management system 24 is responsible for transmitting the alerts and/or notifications 26 to the clinician 28 or a communication device associated with the clinician 28.

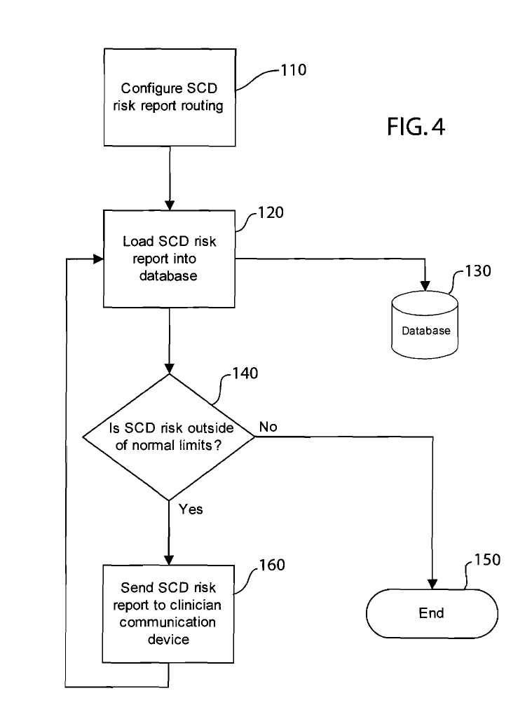

FIG. 4 is a flow chart depicting steps taken by the ECG management system 24 to produce and/or transmit the alerts and/or notifications 26. First, the SCD risk report routing is configured at step 110. If it is determined that the SCD risk report identifies a significant risk, clinician notification is necessary and the SCD report routing identifies the communication devices to which the SCD risk report shall be sent. Next, at step 120 the SCD risk reports are loaded into a database 130. The SCD risk reports are recorded to provide a greater depth of information in the patient's electronic medical history. The SCD risk reports may be recorded whether the risk identified is low risk or high risk. The storage of the SCD risk reports in a database 130 allows for further trending and/or risk analysis to be applied to the data from multiple reports over the course of a patient's care.

Next, the SCD risk reports are analyzed at step 140 to determine if the SCD risk is outside of the normal limits. Alternatively, at step 140 the SCD risk may be gradated as low, medium or high SCD risk or may identify the SCD risk as percentage chance of occurrence. Clinician actions may be taken depending upon the identified SCD risk. Low risk SCD reports result in low priority notification and limited clinician action or in some cases no notification to clinicians. A high risk SCD report may be transmitted to clinicians via high importance or priority communications. This may result in clinicians taking immediate or aggressive action.

If the SCD risk is not determined to be outside of the normal limit then the program ends at step 150 and no indication is sent to the clinician. Alternatively, if it is determined at step 140 that the SCD risk is outside of normal limits, then the SCD risk report is sent to a clinician communication device. The specific communication devices to which the SCD risk report, as well as the format of transmissions may be those that are determined in step 110 in configuring the SCD risk report routing.

Due to the gradation of the SCD Risk report results and predetermined risk report routing procedures, notification of a patient's SCD risk is made to a clinician in a manner commensurate with that risk. Thus, the clinician is not distracted from other duties when the SCD risk is determined to be a low or normal condition while the clinician is made aware if the situation changes to a much more serious risk of SCD occurrence.

While embodiments of the system and method have been disclosed herein, it should be also noted that alternate embodiments of the invention can be in the form of a Holter analysis device that exhibits sudden cardiac death analysis capability. The Holter analysis device typically comprises a series of modules that perform the steps of the method as disclosed herein. Generally speaking, a module includes any implementation in hardware, software, or firmware that performs a specified function. Many modules may receive an input, perform a signal or data processing function on the input, and produce an output; however, this is not limiting to the types of functions that may be performed by a module as disclosed herein.

The Holter analysis device may include a data retrieval module that acquires physiological data at specified time intervals. The physiological data may be, but is not limited to ECG data. The acquired physiological data is processed by a first sudden cardiac death analysis technique module. The first technique module is configured to apply a first sudden cardiac death analysis technique to the acquired physiological data. A first indication of sudden cardiac death risk is produced from the first sudden cardiac death analysis technique module. Next, a second sudden cardiac death analysis technique module is configured to apply a second sudden cardiac death analysis technique to the acquired physiological data. The second technique module receives the physiological data, applies the second sudden cardiac death analysis technique to the acquired physiological data and produces a second indication of sudden cardiac death risk. Further embodiments include additional cardiac death risk analysis technique modules; however, these embodiments are not intended to be limiting on the scope of the Holter Analysis device as disclosed herein.

A sudden cardiac death report generation module receives the first and the second indications of the sudden cardiac death risk and produces a sudden cardiac death report based upon the first and second indications. This sudden cardiac death report is therefore based upon the SCD risk results of applying at least two SCD analysis techniques to the acquired physiological data. This SCD report may be saved in a storage module, or may be transmitted to a clinician or another part of the hospital IT network, such that notification may be made of the results of the sudden cardiac death report.

An alternative embodiment of the Holter analysis device may further include an electrocardiographic data annotation module. This embodiment may be used when the acquired physiological data is ECG data. The data annotation module may include tools for a clinician to use, or be configured to automatedly identify ECG characteristics and morphologies and label these in the collected ECG data.

Embodiments of the systems, methods and devices as disclosed herein may present advantages over current SCD risk determination systems, methods and devices. One advantage is that embodiments as disclosed herein present a predictive or proactive approach to SCD risk analysis. The presently available systems, methods and devices depend upon a lengthy collection of ECG or other physiological data and provide a retrospective analysis of the previously collected data. This results in a reactive approach to patient conditions that previously existed. In many cases this may result in the patient being prematurely discharged from the hospital or inadvertently traveling to a location where medical assistance may be difficult to obtain in the event of a cardiac episode that may lead to SCD. Therefore the concurrent analysis of the patient's ECG and/or other physiological data and computation of SCD risk provides yet another tool for a clinician in analyzing the overall medical health of a patient while under the clinician's care. Additionally, embodiments as disclosed herein provide the advantage of producing a composite SCD risk analysis that utilizes multiple SCD risk analysis techniques and/or algorithms. This provides a more robust indication of SCD risk as weaknesses in single, specific SCD risk algorithms may be overcome by the strength in other algorithm that may be concurrently applied to the collected ECG and/or physiological data.

As disclosed herein, some embodiments of the system, method, and devices may be implemented solely on a computer, in some such embodiments, method steps and/or system blocks may be performed by software operating on a microprocessor wherein the software is configured as a series of modules that receive an input, apply an algorithm or function to the input and produce a resulting output. In these such embodiments, the technical effect is to produce a more proactive and robust indication of a patient's SCD risk to facilitate a clinician's ability to assess the overall health and/or cardiac condition of the patient.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent elements with insubstantial differences form the literal languages of the claims.

What is claimed is:

1. A system for predicting sudden cardiac death from physiological data collected from a patient, the system comprising:
a patient monitor connected to at least one patient, the patient monitor acquiring a plurality of physiological data from the patient, the physiological data comprising at least electrocardiographic data;
a Holter analysis workstation communicatively connected to the patient monitor to acquire the patient physiological data, the Holter analysis workstation applying a plurality of data analysis algorithms to the physiological data to create a sudden cardiac death report; and
a hospital information network that communicatively connects a plurality of clinicians and a plurality of hospital records with the Holter analysis workstation such that hospital records may be updated to include the sudden cardiac death report and at least one clinician may be notified of the results of the Holter analysis workstation.

2. The system of claim 1 wherein the plurality of data analysis algorithms comprises at least T-wave alternans detection, measurement of heart rate turbulence, and measurement of heart deceleration capacity.

3. The system of claim 2 further comprising an electrocardiography management system, the management system forming the communicative connection between the Holter analysis workstation and the hospital information network.

4. The system of claim 3 wherein the patient monitor collects physiological data from the patient in real time.

5. The system of claim 3 wherein the patient monitor collects physiological data from the patient at predetermined time intervals.

6. The system of claim 5 wherein the Holter analysis workstation acquires the cumulative physiological data collected by the patient monitor every twelve hours.

7. The system of claim 3 wherein the electrocardiography management system receives the sudden cardiac death report from the Holter analysis workstation and compares the results of the data analysis algorithms in the sudden cardiac death report to predetermined limits and notifies at least one clinician with an alarm when the in the sudden cardiac death report exceed the predetermined limits.

8. The system of claim 7 wherein the predetermined limits include at least one value range, which when a result is outside the range is indicative of an increased risk of sudden cardiac death.

9. The system of claim 7 further comprising a sudden cardiac death report database communicatively connected to the Holter analysis workstation and the electrocardiography management system; wherein the electrocardiography management system retrieves at least one of a patient's sudden cardiac death reports for determining a patient's risk of sudden cardiac death.

10. The system of claim 9 wherein the electrocardiography management system analyzes a plurality of sudden cardiac death reports when determining a patient's risk of sudden cardiac death.

11. A Holter analysis device with prediction of sudden cardiac death capability, the Holter analysis device comprising:
an electrocardiographic data retrieval module, the module retrieving, at predetermined intervals, electrocardiographic data acquired over a predetermined time period;
a first sudden cardiac death analysis technique module, the first technique module comprising a first configuration module and a first computation module, the first technique module applying a sudden cardiac death analysis technique to the electrocardiographic data to produce a first indication of sudden cardiac death risk;
a second sudden cardiac death analysis technique module, the second technique module comprising a second configuration module and a second computation module, the second technique module applying a sudden cardiac death technique to the electrocardiographic data to produce a second indication of sudden cardiac death risk; and
a sudden cardiac death report generation module that receives the first and second indications of sudden cardiac death risk and produces a sudden cardiac death report based on the first and second indications.

12. The Holter analysis device of claim 11 wherein the first technique is selected from the list of techniques comprising: T-wave alternans detection, measurement of heart rate turbulence, and measurement of heart deceleration capacity, and the second technique is selected from the list of techniques comprising: T-wave alternans detection, measurement of heart rate turbulence, and measurement of heart deceleration capacity.

13. The Holter analysis device of claim 12 further comprising an electrocardiographic data annotation module, the annotation module detecting electrocardiographic morphology and labeling the presence of the detected morphology in the electrocardiographic data.

14. The Holter analysis device of claim 13 further comprising a sudden cardiac death report storage module, the storage module receiving and storing a plurality of sudden cardiac death reports generated for the patient.

15. The Holter analysis device of claim 13 further comprising a third sudden cardiac death analysis technique module, the third technique module comprising a third configuration module and a third computation module, the third technique module applying a sudden cardiac death analysis technique to the electrocardiographic data to produce a third indication of sudden cardiac death risk, wherein the sudden cardiac death report is additionally based on the third indication of sudden cardiac death risk.

16. A method of predicting sudden cardiac death of a patient in a clinical setting, the method comprising:
receiving electrocardiographic (ECG) data from the patient;
applying a first electrocardiographic data analysis technique to generate a first indication of sudden cardiac death risk;
applying a second electrocardiographic data analysis technique to the ECG data, to generate a second indication of sudden cardiac death risk;
analyzing the first indication of sudden cardiac death risk and the second indication of sudden cardiac death risk; and
producing a composite indication of the patient's risk of sudden cardiac death based upon the analysis of the first indication and the second indication.

17. The method of claim 16 wherein the first electrocardiographic data analysis technique and the second electrocardiographic data analysis technique are selected from a list comprising T-wave alternans detection, measuring heart rate turbulence, and measuring heart deceleration capacity.

18. The method of claim 17 further comprising:
comparing the composite indication to at least one predetermined threshold indicative of the patient's risk of sudden cardiac death; and
producing an alarm indicative of the detected risk of sudden cardiac death.

19. The method of claim 17 further comprising applying a third electrocardiographic data analysis technique the third technique being selected from the list comprising: detecting T-wave alternans, measuring heart rate turbulence, and measuring heart deceleration capacity to receive a second indication of sudden cardiac death risk, wherein the composite indication is further based on the results of the third technique.

20. The method of claim 19 further comprising applying at least one additional electrocardiographic data analysis technique selected from the list comprising computing heart rate variability, computing QT interval trends, computing ST interval trends, wherein the composite indication is further based on the results of the at least one additional electrocardiographic data analysis technique.

21. The method of claim 20 further comprising applying at least one non-ECG data analysis technique to other physiological data, wherein the physiological data comprises electrocardiographic data and other physiological data.
